(19) United States
(12) Patent Application Publication
(10) Pub. No.: US 2003/0092666 A1
(43) Pub. Date: May 15, 2003

(54) Compositions and methods for nucleic acid delivery to the lung

(76) Inventors: Mohammed Eljamal, San Jose, CA (US); John S. Patton, San Carlos, CA (US); Linda Foster, Sunnyvale, CA (US); Robert M. Platz, Half Moon Bay, CA (US)

Correspondence Address: Mary Ann Dillahunty, BURNS, DOANE, SWECKER & MATHIS, L.L.P., P.O. Box 1404, Alexandria, VA, 22313-1404, US

(21) Appl. No.: 10/245,722

(22) Filed: Sep. 18, 2002

Related U.S. Application Data
(63) Continuation of application No. 09/978,826, filed on Oct. 16, 2001.
Application No. 09/978,826 is a continuation of application No. 09/665,296, filed on Sep. 20, 2000, now abandoned.
Application No. 09/665,296 is a continuation of application No. 09/427,836, filed on Oct. 26, 1999, now Pat. No. 6,303,582.
Application No. 09/427,836 is a continuation of application No. 08/422,563, filed on Apr. 14, 1995, now Pat. No. 5,994,314.
Application No. 08/422,563 is a continuation-in-part of application No. 08/417,507, filed on Apr. 4, 1995, now abandoned.
Application No. 08/417,507 is a continuation of application No. 08/044,358, filed on Apr. 7, 1993, now abandoned.

(51) Int. Cl. A61K 48/00; A61K 9/16; A61K 9/50

(52) U.S. Cl. 514/44; 424/93.2; 424/493

(57) ABSTRACT

A dry powder composition comprises insoluble nucleic acid constructs dispersed within with a hydrophilic excipient material, where the powder particles have an average size in the range from 0.5 μm to 50 μm. Nucleic acid constructs may comprise bare nucleic acid molecules, viral vectors, or vesicle structures. The hydrophilic excipient material will be selected to stabilize the nucleic acid molecules in the constructs, enhance dispersion of the nucleic acid in dry powder aerosols, and enhance wetting of the nucleic acid constructs as they are delivered to moist target locations within the body.

This application is a continuation-in-part of application Ser. No. 08/______ (attorney docket no. 15225-000410), filed on Apr. 4, 1995, which was a file wrapper continuation of application Ser. No. 08/044,358, the full disclosures of which are incorporated herein by reference.

### BACKGROUND OF THE INVENTION

1. Field of the Invention

The present invention relates generally to compositions and methods for delivering nucleic acids to the lungs of humans and other animal hosts. More particularly, the present invention relates to compositions which are formed by incorporating insoluble nucleic acid constructs within a hydrophilic excipient matrix which is stored and utilized in dry powder form.

A form of human gene therapy which is receiving increasing interest relies on the in vivo delivery of functional nucleic acids, usually structural genes, to certain target cells within a human or other host. The nucleic acids may be incorporated into carriers such as viruses, liposomes, or the like, and will be delivered under conditions which result in uptake of the genes into the target cells, with subsequent expression of the genes for an extended period of time.

Of particular interest to the present invention, it has been demonstrated that nucleic acid constructs can be delivered to the lungs of mice and rats by different routes, including intratracheal administration of a liquid suspension of the nucleic acids and inhalation of an aqueous aerosol mist produced by a liquid nebulizer. Although holding great promise, both methods for the delivery of nucleic acids to the lungs suffer from certain drawbacks. Intratracheal administration is not suitable for routine therapeutic use in humans and has a very low patient acceptability. Moreover, intratracheal instillation often results in very uneven distribution of a dispersion in the lungs, with some regions receiving very little or no material. The use of a liquid nebulizer enjoys higher patient acceptability and achieves better distribution, but requires time-consuming equipment set-up, can require prolonged periods of treatment to achieve an adequate dosage, can inactivate a viral carrier, and can result in undesirable aggregation or degradation of the nucleic acids within the aerosol mist. Aggregated nucleic acids will generally be less suitable for uptake into host target cells.

For these reasons, it would be desirable to provide improved compositions and methods for the aerosol delivery of nucleic acids. The compositions will preferably be in a dry powder form which can be readily dispersed in a flowing air stream to provide a dry aerosol for delivery to a patient. The dry powder formulations will permit delivery of required dosages of nucleic acids in a very rapid manner (typically in several or fewer breaths) and will be suitable for storage over extended periods. The dry powders are delivered to particular target regions within the host and are readily dispersed over the internal surfaces of lung, where the powder dissolves in the moist layer over the surfaces to thereby release nucleic acids to interact with the target cells.

2. Description of the Background Art

Stribling et al. (1992) J. Biopharm. Sci. 3:255-263, describes the aerosol delivery of plasmids carrying a chloramphenicol acetyltransferase (CAT) reporter gene to mice. The plasmids were incorporated in DOTMA or cholesterol liposomes, and aqueous suspensions of the liposomes were nebulized into a small animal aerosol delivery chamber. Mice breathing the aerosol were found to at least transiently express CAT activity in their lung cells. Rosenfeld et al. (1991) Science 252:431-434, describes the in vivo delivery of an α1-antitrypsin gene to rats, with secretion of the gene product being observable for at least one week. The gene was diluted in saline and instilled directly into the rat trachea. Underwood et al. (1991) J. Pharmacol. Meth. 26:203-210, describes the administration of dry powder bronchodilators in a lactose carrier to pig lungs. U.S. Pat. No. 5,049,388 describes the delivery of liquid aerosols containing liposomes to the lungs. Friedman (1989) Science 244:1275-1281 is a review article describing human gene therapy strategies. The presence of certain polyvalent ions can reduce transfection efficiency in vitro using liposomes. Felgner and Ringold (1989) Nature 387-388. Multivalent anions such as citrate or phosphate can induce fusion of positive-charged liposomes used for transfection. Gershon et al. (1993) Biochemistry 32:7143-7151.

### SUMMARY OF THE INVENTION

According to the present invention, dry powder nucleic acid compositions comprise insoluble nucleic acid constructs (typically small particles) dispersed within a matrix of hydrophilic excipient material to form large aerosol particles. Usually, the nucleic acid particles will be present in excess powdered excipient material, usually being the same excipient which forms the matrix. The powdered aerosol particles will have an average particle size in the range from 0.5 μm to 200 μm, usually being in the range from 0.5 μm to 5 μm for lung delivery with larger sizes being useful for delivery to other moist target locations. The nucleic acid constructs may comprise bare nucleic acid molecules, viral vectors, associated viral particle vectors, nucleic acids present in a vesicle, or the like.

The dry powder nucleic acid compositions may be prepared by suspending the insoluble nucleic acid constructs in an aqueous solution of the hydrophilic excipient and drying the solution to produce a powder comprising particles of the nucleic acid construct dispersed within the dried excipient material, usually in the presence of excess powdered excipient. The weight ratio of nucleic acid construct to hydrophilic excipient in the initial solution is in range from 2:1 to 1:100, preferably from 1:1 to 1:10, and the solution may be dried by spraying droplets into a flowing gas stream (spray drying) or by vacuum drying to produce a crude powder followed by grinding to produce a final powder.

In the case of particles intended for lung delivery, having a particle size from 0.5 μm to 5μm, each particle may contain from 10 to 10⁷ nucleic acid constructs, usually from 10² to 10⁵ nucleic acid constructs, and preferably from 10³ to 10⁴ nucleic acid constructs. The constructs may be uniformly or non-uniformly dispersed in each particle, and the particles in turn will often be present in excess powdered excipient, usually at a weight ratio (nucleic acid construct:excipient powder free from nucleic acids) in the range from 1:1, to 1:10³ usually from 1:10 to 1:500.

In a preferred aspect of the present invention, aqueous solutions containing the liposome vesicles as nucleic acid constructs will be substantially free from buffering agents and salts. It has been found that drying, particularly spray drying, of such neutrally charged solutions results in powders having enhanced transfection activity compared to powders formed by drying the same liposome vesicles in buffered solutions. In contrast, aqueous solutions containing viral vectors as the nucleic acid constructs usually will be buffered to enhance stability of the viral vectors.

In a second preferred aspect of the present invention, the dry powder nucleic acid compositions will be prepared by spraying droplets of the liquid solution into a heated gas stream over a short time period, typically 50° C. to 150° C. over a period from 10 msec to 100 msec, in a spray dryer. The resulting powder comprising particles containing nucleic acid constructs (and usually containing powdered excipient free from nucleic acids) will then be collected in a partially cooled environment, typically maintained at 5° C. to 50° C., and thereafter stored at a temperature from 5° C. to 25° C. at a low humidity, typically below 5% RH. It has been found that such collection and storage conditions help to preserve and stabilize the compositions and to enhance transfection efficiency.

Methods for delivering nucleic acid constructs according to the present invention comprise directing the dry powder containing the nucleic acid constructs to a moist target location in a host, where the hydrophilic excipient matrix material of the particles will dissolve when exposed to the moist target location, leaving the much smaller nucleic acid construct particles to freely interact with cells. In a preferred aspect of the present invention, the target location is the lung and the particles are directed to the lung by inhalation.

Compositions of the present invention are particularly advantageous since the hydrophilic excipient will stabilize the nucleic acid constructs for storage. Excess powdered hydrophilic excipient can also enhance dispersion of the dry powders into aerosols and, because of its high water solubility, facilitate dissolution of the composition to deposit the nucleic acid constructs into intimate contact with the target membranes, such as the lung surface membrane of the host.

### BRIEF DESCRIPTION OF THE DRAWINGS

FIGS. 1 and 2 are graphs comparing transfection efficiencies among nucleic acid constructs present in powders, stored liquids, and fresh liquids, as described in detail in the Experimental section.

### DESCRIPTION OF SPECIFIC EMBODIMENTS

The nucleic acid constructs of the present invention will comprise nucleic acid molecules in a form suitable for uptake into target cells within a host tissue. The nucleic acids may be in the form of bare DNA or RNA molecules, where the molecules may comprise one or more structural genes, one or more regulatory genes, antisense strands, strands capable of triplex formation, or the like. Commonly, the nucleic acid construct will include at least one structural gene under the transcriptional and translational control of a suitable regulatory region. More usually, nucleic acid constructs of the present invention will comprise nucleic acids incorporated in a delivery vehicle to improve transfection efficiency wherein the delivery vehicle will be dispersed within larger particles comprising a dried hydrophilic excipient material.

A first type of such delivery vehicles comprises viral vectors, such as retroviruses, adenoviruses, and adeno-associated viruses, which have been inactivated to prevent self-replication but which maintain the native viral ability to bind a target host cell, deliver genetic material into the cytoplasm of the target host cell, and promote expression of structural or other genes which have been incorporated in the particle. Suitable retrovirus vectors for mediated gene transfer are described in Kahn et al. (1992) Circ. Res. 71:1508-1517, the disclosure of which is incorporated herein by reference. A suitable adenovirus gene delivery is described in Rosenfeld et al. (1991) Science 252:431-434, the disclosure of which is incorporated herein by reference. Both retroviral and adenovirus delivery systems are described in Friedman (1989) Science 244:1275-1281, the disclosure of which is also incorporated herein by reference.

A second type of nucleic acid delivery vehicle comprises liposomal transfection vesicles, including both anionic and cationic liposomal constructs. The use of anionic liposomes requires that the nucleic acids be entrapped within the liposome. Cationic liposomes do not require nucleic acid entrapment and instead may be formed by simple mixing of the nucleic acids and liposomes. The cationic liposomes avidly bind to the negatively charged nucleic acid molecules, including both DNA and RNA, to yield complexes which give reasonable transfection efficiency in many cell types. See, Farhood et al. (1992) Biochem. Biophys. Acta. 1111:239-246, the disclosure of which is incorporated herein by reference. A particularly preferred material for forming liposomal vesicles is lipofectin which is composed of an equimolar mixture of dioleylphosphatidyl ethanolamine (DOPE) and dioleyloxypropyl-triethylammonium (DOTMA), as described in Felgner and Ringold (1989) Nature 337:387-388, the disclosure of which is incorporated herein by reference.

It is also possible to combine these two types of delivery systems. For example, Kahn et al. (1992), supra., teaches that a retrovirus vector may be combined in a cationic DEAE-dextran vesicle to further enhance transformation efficiency. It is also possible to incorporate nuclear proteins into viral and/or liposomal delivery vesicles to even further improve transfection efficiencies. See, Kaneda et al. (1989) Science 243:375-378, the disclosure of which is incorporated herein by reference.

Hydrophilic excipient materials suitable for use in the compositions of the present invention will be able to form a dried matrix in which the nucleic acid constructs are dispersed in order to stabilize the nucleic acid molecules during storage, facilitate dispersion of the-nucleic acids in dry powder aerosols, and enhance wetting and subsequent contact of then nucleic acids with the moist target locations within a patient or other treated host. A sufficient amount of hydrophilic excipient will be present to form a dry powder matrix in which the nucleic acids are dispersed, typically being present in the resulting particles at a weight ratio (nucleic acid construct:particle) in the range from 1:1 to 1:1000, usually from 1:10 to 1:500. Suitable hydrophilic excipient materials include those listed in Table 1.

| TYPE OF HYDROPHILIC | |
|---|---|
| MATRIX MATERIAL | EXAMPLES |
| Proteins and Peptides | Human serum albumin; |
| | Collagens; Gelatins; |
| | Lung surfactant |
| | proteins; and |
| | fragments thereof. |
| Hyaluronic acid | Hyaluronic acid. |
| Sugars | Glucose; Lactose; |
| | Sucrose, Xylose; |
| | Ribose; and Trehalose. |
| Sugar alcohols | Mannitol. |
| Oligosaccharides | Raffinose and Stachyose. |
| Other carbohydrates | Dextrans; |
| | Maltodextrans; |
| | Dextrins; |
| | Cyclodextrins; |
| | Maltodextrins; |
| | Cellulose; and |
| | Methylcellulose. |
| Amino acids | Glycine; Alanine; and Glutamate. |
| Organic acids and salts¹ | Ascorbic acid; |
| | Ascorbate salts; |
| | Citric acid; and |
| | Citrate salts. |
| Inorganic salts¹ | NaCl; NaHCO₃; NH₄HCO₃; |
| | MgSO₄; and Na₂SO₄. |

¹The use of organic acids and salts, and inorganic salts, as a matrix material is less preferred in the case of liposomal transfection vesicles, where the salts and acids can interfere with the stability of the vesicle.

The dry powder formulations of the present invention may conveniently be formulated by first suspending the nucleic acid constructs, which are generally insoluble in water, in aqueous solutions of the hydrophilic excipient. The relative amounts of nucleic acid construct and hydrophilic excipient material will depend on the desired final ratio of nucleic acid to excipient. conveniently, the ratio of nucleic acid construct to excipient will be in the range from about 2:1 to 1:100 (nucleic acid:excipient), preferably from 1:1 to 1:10, with a total solids concentration in the aqueous suspension being usually less than 5% by weight, more usually being less than 3% by weight.

In the case of nucleic acid constructs comprising liposomal transfection vesicles, the aqueous solutions are preferably free from polyvalent buffering agents (particularly citrate and phosphate), salts, and other negatively charged species (other than the nucleic acids and in some cases the hydrophilic matrix material), which have been found in some cases to reduce transfection efficiency of the resulting dried powders. It is presently believed that such charged species will interact with the liposomal constructs in a deleterious manner as the compositions are dried.

In the case of nucleic acid constructs comprising viral vectors, it is usually desirable that the aqueous solution be buffered in order to enhance the activity of the viral vectors after drying.

The aqueous solution can then be spray dried under conditions which result in a powder containing particles within a desired size range, typically but not necessarily having a mean particle diameter in the range from about 0.5 μm to 50 μm, with the precise particle size depending on the eventual use. For lung delivery, the particle size will typically be in the range from 0.5 μm to 10 μm, usually being from 0.5 μm to 7 μm, and preferably from 1 μm to 4 μm. The mean particle diameter can be measured using conventional equipment such as a Cascade Impactor (Andersen, Ga.).

Higher total solids concentrations within the aqueous solution will generally result in larger particle sizes. Powders having an average particle size above 10 μm, usually in the range from about 20 μm to 50 μm, can be thus formed, and are particularly useful for nasal, dermal, surgical, and wound applications where it is desired that the powder rapidly settle on a target location.

Dry powders can also be formed by vacuum drying, either at room temperature or under freezing temperatures (lyophilization). Usually, it will be desirable to start with an aqueous solution having higher total solids content, typically above 0.1% by weight, more typically above 0.2% by weight. For smaller particles having a size from 0.5 μm to 10 μm, the liquids will usually have an initial solids content from 0.2% to 1% by weight. For larger particles of 10 μm and above, the solids content will usually be from 15% to 10% by weight. The vacuum drying results in a crude powder which can then be further ground, typically by jet milling, to produce a product having a uniform particle size and a desired particle size, typically within the 1 μm to 50 μm range set forth above.

Specific methods for preparing dry powders of a type which are useful in the present invention are described in copending application Ser. No. 08/______ (attorney docket no. 15225-001400), filed on the same day as the present application, entitled Devices, Compositions and Methods for the Pulmonary Delivery of Aerosolized Medicaments, the full disclosure of which is incorporated herein by reference.

The dry powder compositions of the present invention are suitable for delivery to a variety of target locations within a patient or other treated host, with moist membrane locations, such as the lungs, nasal membranes, mouth, throat, stomach, intestines, vagina, and the like being preferred. The compositions may also be used to deliver the nucleic acid constructs the subcutaneous or intramuscular compartment by dry powder injection, or to open wounds, including surgical wounds, in order to deliver genes to exposed tissue.

In the case of delivery to the lungs, the dry powders will have a mean particle diameter in the range from about 1 μm to 5 μm, and may be efficiently dispersed and delivered in a flowing gas stream for inhalation by the patient or host.

A particularly suitable device for dry powder delivery is described in copending application Ser. No. 07/910,048, assigned to the assignee of the present application, and filed on Jul. 8, 1992, the full disclosure of which is incorporated herein by reference.

The following examples are offered by way of illustration, not by way of limitation.

### EXPERIMENTAL

1. Viral Vector Coated with Mannitol Prepared by Spray Drying

A respirable powder incorporating the human cystic fibrosis transmembrane conductance (CFTR) gene and having a particle diameter from 1 μm to 5 μm is formed as follows. The CFTR gene is linked to the adenovirus (Ad) late promoter, the resulting expression cassette is incorporated into an adenovirus vector, as taught in Rosenfeld et al. (1991) Science 252:431-434. The adenovirus vector has a deletion in the E3 region, thus permitting encapsidation of the recombinant genomic DNA including the CFTR gene. The vector further has a deletion in the Elq region, preventing viral replication.

Sufficient adenovirus vector is added to a phosphate buffered saline solution (0.15 mM NaCl, 2.7 mM KCl, 8.1 mM Na₂PO₄, 1.5 mM KH₂PO₄, pH 7.2) containing 5 mg/ml mannitol at 4° C. to provide approximately 10⁸ plaque forming units (pfu)/ml. The resulting solution is spray dried in a commercially available drier from suppliers such as Buchi and Niro.

After spray drying, the powder is collected and stored at less than 10% relative humidity. The powder may be incorporated into inhalation delivery devices as described in copending application Ser. No. 07/910,048.

2. Plasmid Vector in Liposome Coated with Maltodextrin Prepared by Spray Drying

A respirable powder incorporating the α1-antitrypsin (α1AT) gene and having a particle diameter in the range from 1 μm to 5 μm is formed as follows. A plasmid vector carrying the α1AT gene is prepared as described in Gormon et al. (1982) PNAS 79:6777-6781 and Sambrook et al. (1989), Molecular Cloning: A Laboratory Manual, Cold Spring Harbor Laboratory, Cold Spring Harbor, N.Y. The α1AT gene is fused to the human cytomegalovirus (CMV) immediate early promoter/enhancer element. The plasmid is then purified by alkaline lysis and ammonium acetate precipitation, and the nucleic acid concentration is measured by UV absorption.

Plasmid DNA (0.75 mg/ml) is dispersed in an aqueous solution of double distilled water containing 1.35 mg/ml of DOTMA/DOPE liposomes at a 1:1 molar ratio. The resulting mixture is sonicated for 20 minutes in a water bath. Maltodextrin is added to the mixture after sonication at a concentration of 5 mg/ml. The mixture is then spray dried as described in Example 1.

3. Plasmid Vector in Liposomes, Freeze Dried, and Jet Milled

Plasmid DNA (0.75 mg/ml), prepared as described in Example 2, is mixed with a multilamillar dispersion of cationic fusogenic liposomes (1.5 mg/ml) by gentle agitation at 23° C. for 24 hours in a solution containing 10 mg/ml human serum albumin (HSA). The solution is freeze dried in trays, and the resulting powder is jet milled with high purity nitrogen in a conventional jet mill until a mass median aerodynamic diameter of 1 μm to 4 μm is achieved. The resulting respirable powder is stored at less than 10% relative humidity until it is needed for dispersion in a dry powder device for inhalation.

4. Transfection of Cells with Lipid:DNA Complexes and Adenovirus Vectors

Respirable dry powder aerosols containing lipid:DNA complexes or adenovirus vectors for the delivery of active genes to mammalian cells were prepared and tested. Dispersible dry powders containing either vehicles were made with mannitol and/or glycine as bulking agents and HSA as a surface modifier to help disperse the powders. Transfection activities in CFT1 cells (cells from the airways of cystic fibrosis patients) and virus titers of the resulting powders were measured and compared to liquid controls. The dispersibilities and aerodynamic particle size distributions of select powders that retained their transfection activities were also measured. The transfection activities of the lipid:DNA powders, formulated without buffer, were better than both the liquids they were made of and the freshly prepared liquid formulations. Lipids and DNA were complexed with each other at least 15 minutes prior to cytofection. The titers of the virus in the best powder formulation and its liquid control were 76% and 16% of the expected values, respectively. The dispersibility and the respirable fractions of the selected powders ranged from 40 to 64% and 60 to 80%, respectively. These data demonstrate the ability to obtain respirable and stable dry powder formulations of both cationic lipids complexes and adenovirus delivery systems.

Materials and Methods

Lipids:

1. DMRIE:DOPE (50/50, mole ratio, Vical, San Diego, Calif.). The lipids (DMRIE:DOPE) were formulated to generate 1.56 mM solution by resuspending 5 mg vial in 2.4 ml de-ionized water and vortexing at full speed for 1 minute.

2. DOTMA/DOPE (50:50, mole ratio, Megabios, San Francisco, Calif.).

DNA Plasmid:

1. pCMVβ (Genzyme, Framingham, Mass.). pCMV-β-gal: Cytomegalovirus promoter was linked to the Escherichia coli Lac-Z gene, which codes for the enzyme β-galactosidase. The activity of this enzyme was visualized with the reagent X-gal (b-D-galactoside). The DNA plasmid (pCMVβ, 4.26 mg/ml) was formulated to generate 960 μM by adding 0.145 ml of the DNA suspension to 1.9 ml 1 mM tris buffer, pH 8.

2. pCIS-CAT (Megabios, San Francisco, Calif.). pCIS-CAT: Chloramphenicol acetyltransferase (CAT) fused to the human cytomegalovirus (CMV) immediate early promoter/enhancer element.

Lipid:DNA Complex: The complex was formed by first adding DNA plasmids (pCMVβ) to a certain volume of bulking and excipient materials solution to attain the desired concentration then the preformed lipids (DMRIE:DOPE) were added to form the complex at least 10 minutes prior to processing into powder. The lipid:DNA ratio was molar.

Virus: Ad2-CMV-LacZ-2 (Genzyme, Framingham, Mass.). AD2-CMV-Lac-Z: Cytomegalovirus promoter was linked to the Escherichia coli Lac-Z gene and was incorporated into replication deficient recombinant virus. Takiff et al. (1984) J. Virol. 51:131-136 and Gilardi et al. (1990) FEBS Lett. 267:60-62.

1 mM Tris buffer pH 8 (0.14 mg/ml solids): (1) Dissolved 60.6 mg Tris base (J T Baker, lot # x171-07) in 500 ml deionized house water to make a 1 mM solution. (2) Dissolved 78.8 mg Tris HCl (J T Baker, lot # 4103-1) in 500 ml deionized house water to make a 1 mM solution. To the magnetically stirred Tris base solution, Tris HCl was slowly added to obtain pH 8.

Tris/Mannitol/HSA (5.07 mg/ml solids): Dissolved 1,363.0 mg mannitol (Mallinckrodt, lot # 6208 KLRP) and 156.7 mg HSA (Miles, lot # 204) in 300 ml of the 1 mM Tris buffer.

Glycine/HSA (I) (5.44 mg/ml solids): Dissolved 60.6 mg HSA and 1,028.0 mg glycine (J T Baker, Lot # A28732) in 200 ml filtered and deionized house water, pH 6.4.

Glycine/Mannitol/HSA (5.57 mg/ml solids): Dissolved 50.6 mh HSA, 540.0 mg glycine and 524.0 mg mannitol in 200 ml of filtered and deionized house water, pH 6.4.

Phosphate buffer (PB) pH 7.4 (1.89 mg/ml solids): Dissolved 200.1 mg KCl (J T Baker, Lot No. 3040-01), 1,451.4 mg Na₂HPO₄.7H₂O (Mallinckrodt, Lot No. 7896 KJPE) and 242.1 mg KH₂PO₄ (J T Baker, Lot No. 3246-01) in one liter of the house deionized water to make pH 7.4.

Phosphate/HSA (3.93) mg/ml solids): Dissolved 203.8 mg HSA (Miles, Lot No. 204) in 100 ml of the phosphate buffer pH 7.4.

Mannitol/HSA in PB (60.05 mg/ml solids): Dissolved 1,403.1 mg mannitol (Mallinckrodt, Lot No. 6208 KLRP) in 25 ml phosphate/HSA. Stored below 5° C.

Glycine/HSA (I) in PB (28.40 mg/ml solids): Dissolve 611.8 mg glycine (J T Baker, Lot No. 0581-01) in 25 ml phosphate/HSA. Stored below 5° C.

Glycine/HSA (II) in PB (10.5 mg/ml solids): Dissolved 613.8 mg glycine (J T Baker, Lot No. 0581-01) and 1 ml (250 mg) HSA (Alpha Therapeutic, lot # NB2049A) in 100 ml phosphate/HSA. Stored below 5° C.

Glycine/HSA (II) in water (8.6 mg/ml solids): Dissolved 612.4 mg glycine (J T Baker, Lot No. 0581-01) and 1 ml (250 mg) HSA (Alpha Therapeutic, lot # NB2049A) in 100 ml de-ionized water. Stored below 5° C.

Mannitol/Glycerine/HSA in PB (45.09 mg/ml solids): Dissolved 700.2 mg mannitol (Mallinckrodt, Lot No. 6208 KLRP) and 328.8 mg glycine (J T Baker, Lot No. 0581-01) in 25 ml of phosphate/HSA. Stored below 5° C.

Adenovirus (40.20 mg/ml): Dissolved 305.3 mg sucrose (Sigma, Lot No. 69F0026), 77.9 mg NaCl (VWR SCI., Lot No. 34005404) and 0.1 ml of Ad2-CMV-LacZ virus (10¹¹ iu/ml with particle concentration of ⁻5×10¹²/ml in PBS+3% sucrose, Genzyme) in 10 ml phosphate buffer. This solution was prepared and used cold on the same day and was stored frozen at −70° C. Also, it was used again 10 weeks later, it underwent only one freeze/thaw cycle.

Powder processing: All the powders were processed in a Buchi-190 mini spray dryer. Briefly, the solution is atomized into liquid droplets and is dried to solid particulate with adjunct stream of air heated to a specified temperature (inlet temperature). The airborne particulate are fed into a cyclone (outlet temperature) where they are separated from the air into a collection cup.

Dispersibility: Dispersibility of the dry powder was determined using a dry powder inhaler (generally as described in application Ser. No. 08/309,691, the full disclosure of which is incorporated herein by reference) or a test bed. Briefly, a blister pack filled with 5.0±0.5 mg powder was loaded and dispersed in the device. The resulting aerosol cloud in the device chamber was immediately drawn at a suction flowrate of 30 LPM for 2.5 seconds and was collected on a 47 mm, 0.65 μm pore size, polyvinylidene fluoride membrane filter (Millipore). Dispersibility is the fraction of powder mass collected on the filter relative to mass filled into the blister pack.

Particle size (Horiba): The particle size distribution (PSD) of the powder samples was measured using the Horiba CAPA-700 centrifugal sedimentation particle size analyzer. Approximately five mg of powder was suspended in approximately 5 ml of Sedisperse A-11 (Micromeritics, Norcoss, Ga.) and briefly sonicated before analysis. The instrument was configured to measure a particle size range of 0.4 to 10 μm in diameter, and the centrifuge was operated at 2000 rpm. The particle size distribution was characterized by mass median diameter, and by the mass fraction less than 5.0 μm.

Particle size (cascade impactor): The particle size distribution of aerosolized powders (aerosol from blister using prototype 1B device) was obtained using an IMPAQ 6-stage (16, 8, 4, 2, 1, 0.5 μm cut off diameters) cascade impactor (California Measurement, Sierra Madre, Calif.). A glass Throat, described in the European Pharmacopoeia, was fitted over the intake of the cascade impactor. The glass throat was designed to simulate particle deposition in the human throat when aerosol is sampled in the cascade impactor. The impactor airflow was set to 14.5 LPM, the calibrated operating flow of the instrument. To measure the particle size of the aerosol, a blister pack filled with approximately 5 mg of powder was loaded into the prototype inhaler, the device was actuated and the aerosol cloud drawn from the chamber into the glass throat/cascade impactor set-up. The particle size was determined gravimetrically by weighing the powder on the glass throat, impactor plates and the backup filter and plotting the results on a log-probability graph. The mass median aerodynamic diameter (MMAD) and the mass fraction less than 5 μm were determined from the graph.

Lipid:DNA Gene Therapy

Cationic Liposomes Dry Powder

The following formulations were made to develop aerosol liposomes in dry powder format. Cationic lipid (34.5 mg (25 μMoles) DOTMA:DOPE, 1:1, Megabios) was dispersed in 100 ml of 6.75 mg/ml mannitol solution. This solution (7.1 mg/ml solids) was processed into powder according to the following spray drying parameters:

Solution feed rate: 5.8 ml/min

Inlet/Outlet Temperatures: 137/73° C.

Atomizer air flow rate: 800 LPH

The powder yield was about 6% and could not be filled into blister packs. The resulting powder was sticky, possibly due to liposomes presence on the surface of the powder. This possibly resulted from the cationic liposomes on the surface of the dry particles strongly interacting with each other. In order to solve this problem, Human serum albumin (HSA) in solution to increase the dispersibility of the powder by modifying its surface morphology.

Two liquid formulations containing HSA (Alpha Therapeutic, 12.5 g/50 ml solution), lipids (DOTMA:DOPE) and mannitol were dried in the Buchi-190 spray dryer. The liquid solution was fed at 3 ml/min and the inlet/outlet temperatures ranged between 95-105° C./55-70° C. We found that both the yield and the dispersibility of the dry powder was improved with the addition of HSA (see Table 1).

TABLE 1
Summary of Lipids/Mannitol aerosol formulations.

| | Composition | | |
|---|---|---|---|
| | HSA/Lipids/Mannitol | Yield | Dispersibility |
| Formula No. | (mg/ml) | Percent | Percent |
| 1 | 0.00/0.35/6.75 | 6 | — |
| 2 | 0.40/0.35/6.40 | 55 | 36 ± 6 |
| 3 | 0.91/0.35/6.40 | 54 | 59 ± 4 |

DNA Powder

Experimental

To investigate whether this process would preserve the integrity of DNA molecules, pCMVβ in Tris/Mannito/HSA solution (7.5 mg/ml solids) was spray dried according to the following conditions:

Solution feed rate: 4.3 ml/min

Inlet/Outlet Temperatures: 120° C./70° C.

Atomizer air flowrate: 800 LPH

The resulting powder was reconstituted in de-ionized water and was run in gel electrophoresis (1.3% agrose in 0.5×TBE plus 0.5 μg/ml ethidium bromide, 100 volts for four hours). Unprocessed DNA molecules were also run in the same gel. The powder was tested for transfection activity in vitro as follows:

Cytofection Assay

Cell Preparation:

Cells of choice (CFT1, airway cells from cystic fibrosis patients) were placed into 96-well plates at 20,000/well in growth medium the day before cytofection. Just prior to cytofection, the cells were observed, and approximate confluencey estimated.

Lipid:DNA Preparation:

The lipid was formulated to 670 mM and the DNA to 960 mM. The complex was formed by adding the lipid to the DNA for 15 minutes, and then 100 μl of the complex was added to the cells (media previously aspirated). Cytofection occurred over 6 hours before the addition of 50 μl 30% FCS-OPTIMEM. The following day, 100 μl of 10% FCS-OPTIMEM was added to each well. The assay began 48 hours after start of cytofection.

Assay:

1. Remove media and wash cells twice with 100 μl PBS

2. Add 25 μl lysis buffer (250 mM Tris-HCl, pH8.0, and 0.15% Triton X-100) and incubate at RT for 30 minutes.

3. Freeze plate at −70° C. for 20 minutes, thaw at RT for 15 minutes.

4. Break up cells by carefully vortexing plate for 15 seconds.

5. Freeze plate at −70° C. for 20 minutes, thaw at RT for 15 minutes.

6. Add 100 μl PBS followed by 150 μl of CPRG substrate (1 mg/ml chlorophenol red glactopyranoside, 60 mM disodium hydrogen phosphate pH8, 1 mM magnesium sulfate, 10 mM potassium chloride, and 50 mM β-mercaptoethanol)

7. Incubate at 37° C. for 2 hrs until red color develops and read at 580 nm in microplate reader.

Results

Similar bands were observed for both processed and unprocessed DNA in the gel electrophoresis. As expected the reconstituted DNA (without any delivery vehicle, cationic lipid or adenovirus) powder did not show any transfection activity.

Lipid:DNA Powder

Experimental

Three sets of cationic lipid:DNA formulations were prepared, processed into dry powder and characterized:

1. The lipid:DNA complex was formed in Tris/mannitol/HSA solution (5.07 mg/ml solids) with the following concentration ratios of lipid:DNA (μM:μM)-0:0, 0:6.9, 20.9:12.8, 10.4:12.8, 5.2:12.8, 10.4:6.9, 5.2:6.9, 2.6:6.9, 0.4:3.5, 5.2:3.5 and 2.6:3.5.

2. The lipid:DNA complex was formed in glycine/HSA (I) in water (5.44 mg/ml solids) with the following lipid:DNA concentration ratios (μM:μM)-20:20, 20:15, 10:10 and 10:5.

3. The lipid:DNA complex was formed in glycine/mannitol/HSA solution (5.57 mg/ml solids) with the following ratios (μM:μM)-20:20, 20:15, 10:15, 10:10 and 10:5. The solutions were processed into powder according to the following spray drying parameters:

Solution feed rate: 3.8 ml/min

Inlet/Outlet Temperatures: 115-125° C./70-85° C.

Atomizer air flowrate: 700-800 LPH

Aliquots of the liquid formulations and the resulting powders were kept refrigerated and duplicates were sent on ice pack to be assayed for transfection activity in vitro (as described above) and also to be compared with freshly prepared suspensions of Lipid:DNA with similar concentration ratios. Select powders from sets 2 and 3 were characterized using the Horiba, IMPAQ 6-stage cascade impactor and a dry powder inhaler.

Results

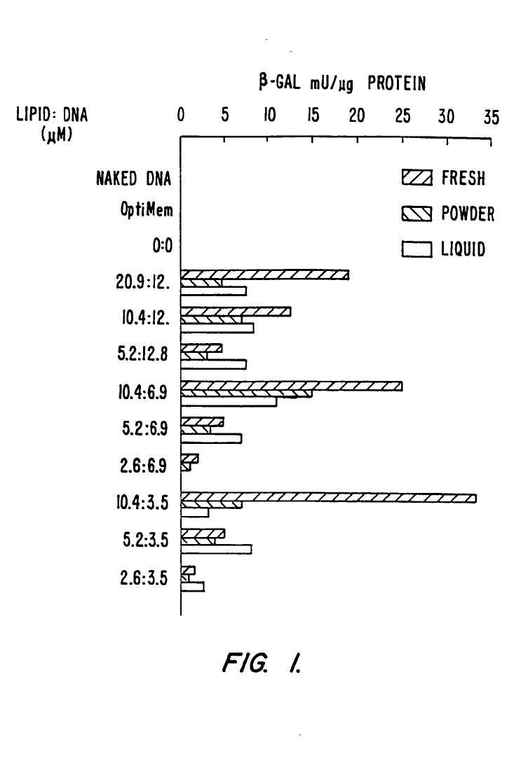

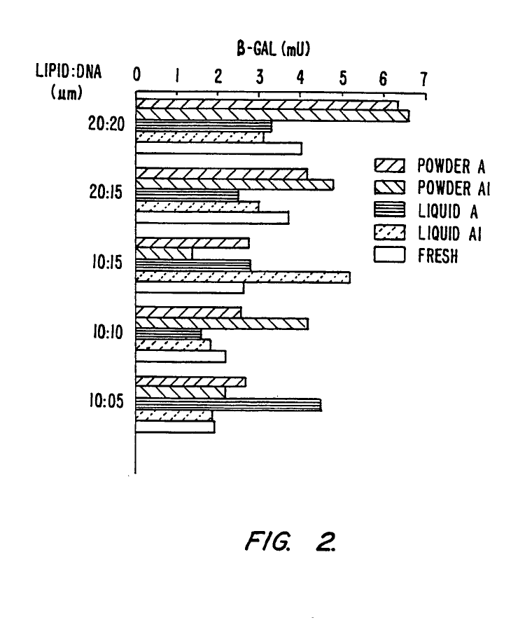

A comparison of β-gal expression in vitro (CFT1 cell line) between the powder and the two liquid (stored control and freshly made control) formulations are shown in FIG. 1 and 2. The powders were reconstituted in double distilled de-ionized water. The transfection activities of the liquid and powder formulations of set 1, which contained the Tris buffer, were considerably less than freshly made liquid formulations (FIG. 1). In the powders, which contained no buffer, there was a 75% increase in the transfection activity of the 20:20 and 30% increase in the 20:15 as compared with liquid formulations (see FIG. 2). The measured physical parameters of the selected powders that showed superior transfection are listed in Table 2. The glycine/HSA and glycine/mannitol/HSA powder formulations had similar transfection activities (FIG. 1) but the glycine/HSA powders dispersed better than the glycine/mannitol/HSA (Table 2).

TABLE 2
Lipid: DNA powder physical characteristics.

| | | Dipersi. | | | |
|---|---|---|---|---|---|
| Formula | Bulking | (% RSD) | HORIBA | Cascade | Impactor |
| ratio | Material | (n = 3) | MMD* | MMAD** | % ≦ 5 μm |
| 20:20 | Glycine | 61 (20) | 2.0 | 3.9 | 60 |
| 20:15 | Glycine | 64 (1) | 2.0 | 2.4 | 75 |
| 20:20 | Gly/Man | 47 (12) | 2.0 | 3.0 | 70 |
| 20:15 | Gly/Man | 51 (12) | 2.4 | 4.1 | 60 |

*MMD: Mass Median Diameter
**MMAD: Mass Median Aerodynamic Diameter

Adenovirus Gene Therapy: Dry Powder Aerosol Development Experimental

This developmental study included two sets of experiments. In the first set, the effects of bulking agents in phosphate buffer (PB), (i) mannitol/HSA, (ii) glycine/HSA and (iii) mannitol/glycine/HSA, on the infectivity of the adenovirus dry powders were investigated. In the second set, we investigated the effects of buffer removal and the process outlet temperature on the infectivity. All solutions were used and stored cold (about 5° C.).

1. Five mannitol/HSA in PB formulations were prepared: (i) To 4×3 ml mannitol/HSA in we added 0.1 ml of adenovirus solution to obtain 3.2×10⁷ iu/ml and about 60 mg/ml solids, and the fifth was used as a control with no virus. Two of the virus formula were diluted with de-ionized water to about 9 mg/ml solids. (ii) Two formulations of 6.3 ml glycine/HSA (I) in PB plus 0.4 ml adenovirus solution were made (29 mg/ml solids, 6.3×10⁷ iu/ml). One of them was diluted with de-ionized water to 9 mg/ml solids. (iii) Two formulations of 4.1 ml mannitol/glycine/HSA in PB plus 0.4 ml of virus solution were made (45.1 mg/ml solids, 8.89×10⁷ iu/ml). One was diluted with de-ionized water to 9 mg/ml. The adenovirus solution was freshly made on the same day and was kept cold on ice.

2. Four formulations were prepared, two contained 25 ml of glycine/HSA (II) in PB plus 0.4 ml of adenovirus solution (10.5 mg/ml, 1.6×10⁷ iu/ml) and the other two contained 25 ml of glycine/HSA (II) in water plus 0.4 ml of adenovirus solution (8.6 mg/ml, 1.6×10⁷ iu/ml). The adenovirus solution underwent only one freeze/thaw cycle before usage in the above preparations. It was prepared around 10 weeks ago and was stored frozen at −70° C.

These formulations were processed into powders in the Buchi-190 spray dryer according to the following parameters:

Solution feed rate: 3.5-6.0 ml/min

Inlet/Outlet temperatures: 100-140/70-90° C.

Atomize flowrate: 700-800 LPH

The resulting powder was kept refrigerated and was sent for testing on dry ice. Prior to testing for β-gal expression or for virus titers, the powders were reconstituted with phosphate buffered saline (PBS).

Results

None of the mannitol powder formulations showed any β-gal expression in the standard 6-well test and therefore they were not titered for virus infectivity. The glycine/HSA (I) and glycine/mannitol/HSA in PB from set one were equal in their β-gal expression and were tittered for virus infectivity. Their titers ranged from 7% to 15% of the expected values. The particle size distribution (HORIBA), dispersibility and the aerodynamic size distribution (IMPAQ 6-stage) are listed in Table 3 for the two glycine/HSA in PB powders.

Set two powders and 0.1 ml of the adenovirus solution (V) frozen to −70 C. were sent on dry ice for titer measurements (Table 4). Powders manufactured with and without the phosphate buffer retained 76-54% and 2-1.4% of their virus infectivities, respectively (Table 4). Lowering the outlet temperature by 5° C. increased the buffered formulation virus infectivity by 22% but it lowered the unbuffered one by 6%.

TABLE 3
Glycine/HSA adenovirus formulations.

| Formula | Dipersi. | HORIBA | Cascade impactor | | % infectivity |
|---|---|---|---|---|---|
| (mg/ml) | (% RSD) | MMD | MMAD | % < 5μm | retained |
| 29 | 40 (25) | 2.6 | 2.8 | 70 | 14 |
| 9 | 51 (1) | 2.3 | 1.8 | 80 | 7 |

TABLE 4
Adenovirus powders in buffer and without buffer titer results.

| | Outlet Temp. | Expected | Measured |
|---|---|---|---|
| Formulation | ° C. | iu/ml | iu/ml |
| V | N/A | 1.0 X 10⁹ | 1.6 X 10⁸ |
| Buffered | 77 | 1.0 X 10⁸ | 5.4 X 10⁷ |
| Buffered | 72 | 1.0 X 10⁸ | 7.6 X 10⁷ |
| Unbuffered | 77 | 1.0 X 10⁸ | 2.0 X 10⁶ |
| Unbuffered | 72 | 1.0 X 10⁸ | 1.4 X 10⁶ |

Although the foregoing invention has been described in some detail by way of illustration and example, for purposes of clarity of understanding, it will be obvious that certain changes and modifications may be practiced within the scope of the appended claims.

What is claimed is:

1. A dry powder nucleic acid composition comprising insoluble nucleic acid constructs dispersed within a hydrophilic excipient material.

2. A dry powder nucleic acid composition as in claim 1, wherein the composition consists essentially of particles of the nucleic acid constructs dispersed within the hydrophilic excipient material, present in a powder of the excipient material.

3. A dry powder nucleic acid composition as in claim 2, wherein the nucleic acid construct particles have an average particle size in the range from 0.5 μm to 50 μm.

4. A dry powder nucleic acid composition as in claim 1, wherein the nucleic acid constructs comprise bare nucleic acid molecules or viral vectors.

5. A dry powder nucleic acid composition as in claim 1, wherein the nucleic acid constructs comprise nucleic acids present in a vesicle, wherein the vesicle is dispersed within the hydrophilic excipient.

6. A dry powder nucleic acid composition as in claim 1, wherein nucleic acid construct includes a structural region and a regulatory region.

7. A dry powder nucleic acid composition as in claim 1, wherein the hydrophilic excipient is a material selected from the group consisting of inorganic salts, sugars, sugar alcohols, oligosaccharides, amino acids, organic acids and salts, carbohydrates, proteins, and peptides.

8. A method for preparing dry powder nucleic acid compositions, said method comprising:
suspending insoluble nucleic acid constructs in an aqueous solution of a hydrophilic excipient; and
drying the solution to produce a powder comprising particles of the nucleic acid constructs dispersed within the dried excipient material.

9. A method as in claim 8, wherein the nucleic acid constructs are present in the aqueous solution at a weight ratio in the range form 2:1 to 1:100 nucleic acid construct: hydrophilic excipient.

10. A method as in claim 8, wherein the aqueous solution is dried by spraying droplets into a gas stream, wherein particles having a size in the range from 0.5μ to 50 μm are produced.

11. A method as in claim 8, wherein the aqueous solution is dried by exposure to a vacuum to produce a crude powder, further comprising grinding the crude powder to produce a final powder size in the range from 1 μ to 50 μm.

12. A method as in claim 8, wherein the nucleic acid constructs comprise bare nucleic acid molecules or viral vectors and the aqueous solution is buffered.

13. A method as in claim 8, wherein the nucleic acid constructs comprise nucleic acids present in a vesicle and the aqueous solution is substantially free from buffer and salts.

14. A method as in claim 8, wherein the nucleic acid constructs include a structural region and a regulatory region.

15. A method as in claim 8, wherein the hydrophilic excipient is a material selected from the group consisting of inorganic salts, sugars, sugar alcohols, oligosaccharides, amino acids, organic acids and salts, carbohydrates, proteins, and peptides.

16. A method for delivering nucleic acid constructs to a moist target location in a patient, said method comprising
dispersing a dry powder comprising particles of insoluble nucleic acid constructs in a hydrophilic excipient material in a gas stream; and
directing the gas stream at the moist target location, whereby the hydrophilic excipient coating absorbs water and dissolves to release the nucleic acid constructs.

17. A method as in claim 16, wherein the target location is the lung and the gas stream is directed to the lung by inhalation.

18. A method as in claim 17, wherein the coated nucleic acid constructs have a particle size in the range from 0.5 μm to 50 μm.

19. A method as in claim 16, wherein the nucleic acid constructs comprise bare nucleic acid molecules or viral vectors.

20. A method as in claim 16, wherein the nucleic acid constructs comprise nucleic acids present in a vesicle.

21. A method as in claim 16, wherein the nucleic acid constructs include a structural region and a regulatory region.

22. A method as in claim 16, wherein the hydrophilic excipient is a material selected from the group consisting of inorganic salts, sugars, sugar alcohols, oligosaccharides, amino acids, organic acids and salts, carbohydrates, proteins, and peptides.
